(19) United States
(12) Patent Application Publication
(10) Pub. No.: US 2011/0098709 A1
(43) Pub. Date: Apr. 28, 2011

(54) MECHANICAL CAVITY-CREATION SURGICAL DEVICE AND METHODS AND KITS FOR USING SUCH DEVICES

(75) Inventors: Hugues F. Malandain, Mountain View, CA (US); Lex Philip Jansen, Pleasanton, CA (US)

(73) Assignee: KYPHON SARL, Neuchatel (CH)

(21) Appl. No.: 12/984,551

(22) Filed: Jan. 4, 2011

Related U.S. Application Data
(62) Division of application No. 11/454,525, filed on Jun. 16, 2006.
(60) Provisional application No. 60/727,093, filed on Oct. 14, 2005.

(51) Int. Cl. A61B 17/32

(52) U.S. Cl. 606/79

(57) ABSTRACT

A mechanical cavity-creation surgical device and methods and kits for using such devices is described. In one variation, the mechanical cavity-creation surgical device contains a side window at the distal end of a shaft that allows the articulation of a blade, pick, or tip from a position that is inside the shaft to a position that protrudes at least partially from the side window.

This application claims priority under 35 U.S.C. §119(e) to U.S. Provisional Patent Application No. 60/727,093 filed Oct. 14, 2005, the entire contents of which are incorporated herein by reference.

### FIELD OF THE INVENTION

The present invention relates to a mechanical cavity-creation surgical device and methods and kits for using such devices, as well as methods for making such devices.

### BACKGROUND

One challenge in designing a curette of small size for surgical repair of internal body structures, such as bone, is to manufacture a curette that is small enough to fit in a cannula, but that is strong enough so that the curette is not damaged during the process of deploying the curette to the site being repaired, or while the curette is being used to scrape or score the surgical site. Because of the small size of the curette, there may be a need for a deployable curette tip that can be hidden within the shaft for low profile insertion into a patient's body.

### SUMMARY OF THE INVENTION

A mechanical cavity-creation surgical device and methods and kits for using such devices is described. In one variation, the mechanical cavity-creation surgical device contains a side window (e.g., a slot or aperture) at the distal end of a shaft that allows the articulation of a blade, pick, or tip from a position that is inside the shaft to a position that protrudes at least partially from the side window.

### BRIEF DESCRIPTION OF THE DRAWINGS

FIG. 1 shows a perspective three-dimensional view of a curette that exemplifies one embodiment of the present invention.

FIGS. 2A and B show a transparent three-dimensional rendering of the lumen of the curette of FIG. 1 and a cross-sectional top down view of the lumen of the curette of FIG. 1, respectively.

FIG. 3 shows a transparent three-dimensional rendering of the shaft of the curette of FIG. 1 wherein the blade and the mode of action for moving the blade of the curette is visible.

FIGS. 4A-D show transparent 3D views of the blade in the lumen of the curette of FIG. 1 at different levels of deployment of the blade.

### DETAILED DESCRIPTION

The present invention relates to curettes that are designed for minimally invasive surgery. In one embodiment of the invention, the curette is designed to pass through a cannula. Alternatively, the curette does not pass through a cannula and the shaft of the curette can serve as a cannula. Embodiments of curettes according to the present invention may be designed to have a small diameter shaft with a lumen on the interior of the shaft, a handle at the proximal end of the shaft, and a blade and/or cutting tip at the distal end of the shaft, where the blade deploys from a first position that is inside the lumen of the shaft and parallel to the length of the shaft, to a second position that is at least partially outside of the shaft and that is not parallel to the length of the shaft. In an embodiment, when the blade and/or tip is at its furthest deployed or articulated position, the blade (or tip) may be axial (or perpendicular) to the shaft. The blade may be positioned inside the shaft during deployment/insertion of the curette so as to prevent premature splaying of the tip or blade while the shaft/curette is being positioned at a surgical site. The blade or tip of the curette can be of sufficient length and strength so as to adequately scrape or score the sides of a body cavity to generate a cavity of sufficient size when articulated to the desired position. Alternatively, a plurality of blades of various sizes may be used.

As used in this written description and the appended claims, the singular forms “a,” “an” and “the” include plural referents unless the context clearly dictates otherwise. Thus, for example, the term “a lumen” is intended to mean a single lumen or a combination of lumens. Furthermore, the words “proximal” and “distal” refer to directions closer to and away from, respectively, an operator (e.g., surgeon, physician, nurse, technician, etc.) who would insert the medical device into the patient, with the tip-end (i.e., distal end) of the device inserted inside a patient's body. Thus, for example, the end of a medical device inserted inside the patient's body would be the distal end of the medical device, while the end of the medical device outside the patient's body would be the proximal end of the medical device.

The shaft may have a proximal end (closest to the user) and a distal end (closest to the surgical site). In one embodiment, the proximal end of the shaft may comprise a handle. Additionally or alternatively, the distal end of the shaft may comprise a cap or other type of enclosure.

The length of the shaft may be varied depending upon the required procedure and the body structure to be accessed. In alternate embodiments, the shaft length may range from about 20 to 40 cm, or from about 25 to 35 cm, or from 25 to 30 cm. Thus, the shaft length can be any desired length so as to achieve the intended purpose (i.e., it can be very short when the surgical site is close or longer when the surgical site is further away).

The shaft (e.g., from the handle at the proximal end to the cap at the distal end) may, in certain embodiments, comprise a substantially uniform diameter. For example, in alternative embodiments the outer diameter (OD) of the shaft may comprise a diameter of about 0.1 to about 2.5 cm, or from about 0.1 to about 0.5 cm, or from about 0.2 to about 0.4 cm in diameter or from about 0.25 to about 0.35 cm in diameter. In one embodiment, the diameter may range from about 0.109 inches (size 2=˜0.28 cm) to 0.134 inches (size 3=˜0.35 cm). Although example ODs have been given above, one of ordinary skill in the art having the benefit of this disclosure would appreciate that the shaft OD can be designed to accommodate the specific surgical purpose of the curette.

The inner diameter of the shaft will be smaller than the outer diameter, but may comprise a cylinder having sufficient inner diameter (ID) space on the inside of the shaft (i.e., the lumen) so as to accommodate the blade. Also, the shaft may comprise a cylinder having sufficient inner diameter (ID) space to accommodate moving parts that allow the blade and/or tip to articulate from a position that has the blade and/or tip in the lumen and parallel to the length of the shaft to a position where the blade and/or tip is at least partially outside the shaft and not parallel to the length of the shaft. For example, in alternative embodiments the inner diameter (ID) of the shaft may comprise a diameter of about 0.09 to 2.4 cm, or from about 0.09 to 0.45 cm, or from about 0.18 to 0.35 cm in diameter. In one embodiment, the inner diameter may range from less than 0.109 inches to less than 0.134 inches.

The shaft may be made from a metal or metal alloy that is suitable for surgical procedures. However, any materials that are satisfactory for surgical purposes may be used. Thus, in an embodiment, the shaft may be made of stainless steel, various nickel-titanium alloys (such as NITINOL), as well as other metal alloys that may be suitable for surgical purposes. It is contemplated that the shaft may be constructed of a hard plastic that may be alternatively, reusable or disposable.

In an embodiment, the handle at the proximal end of the shaft may comprise a control mechanism that allows a user (e.g., a physician or surgeon) to articulate the blade or tip to position near the distal end of the shaft. In this way, the user may control the position of the curette tip or blade during the steps of deployment and/or scraping. Thus, in one embodiment, the handle may be used to articulate the curette tip from a first position that is present in and parallel to the lumen of the shaft, to a desired second articulated position that is at least partially axial and at least partially outside the shaft. The tip or blade can be articulated to any of a plurality of articulated positions with this plurality of positions (i.e., essentially an infinite number of positions) being from a position that is only slightly non-parallel to the shaft (i.e., an angle that is only marginally larger than 0°) to a position that is substantially perpendicular to the shaft (90°). In an alternate variation of this embodiment, the tip, blade or pick may actuate up to 60 degrees. At any one of these plurality of positions, the curette of the present invention may allow for the articulated tip to be stable to torsional strain. Thus, once deployed, the tip may comprise adequate strength to perform its function, such as to scrape or score material from a bone to form a cavity in the bone.

It is contemplated and within the scope of the invention that any of a plurality of control mechanisms can be used to articulate the shaft including a mechanism that the user can manipulate by using his/her hands, or alternatively, may be articulated by using a motor, or some combination of the two. For example, in alternate embodiments, the means by which a user may articulate the curette tip using any mechanical means used for moving two parts in relation to each other such as a mechanical means (e.g. a thumb screw), hydraulic means, pneumatic means, or electromagnetic (e.g., motor) means.

The articulated tip may also have associated with it a torque limiting device that includes similar features that are present in co-pending U.S. patent application Ser. No. 11/256,036, filed Oct. 21, 2005, which is incorporated herein in its entirety by reference for all purposes.

Thus, the handle may be connected to the shaft so that when the handle (or some part thereof) is rotated, the shaft also rotates. In an embodiment, the tip and the shaft are aligned such that the tip and/or blade of the curette may also rotate when the handle and shaft are rotated. The shape of the handle may be such that it allows a physician to easily rotate the handle (and consequently, also rotate the shaft and tip and/or blade) so as to allow the physician to score and/or scrape at the surgical site. Alternatively, the handle may also have adaptations that allow the handle to be connected to a motor that allows the handle (and consequently, also the shaft and the tip and/or blade) to be rotated, creating the desired cavity.

In an embodiment, the distal end of the shaft is at least partially encapsulated. In an embodiment, the distal end of the shaft may comprise a side window (for example, an aperture or slot or any parallel or circumferential opening on the side of the shaft) that allows the curette tip to extend from the shaft. For example, in one embodiment, at or near the distal end of the shaft, there is a slot parallel to the length of the shaft that allows passage of the blade and/or tip from a first position where the blade and/or tip is inside and parallel to the length of the shaft to a position that is at least partially outside the length of the shaft and not parallel to the shaft. The aperture at the distal end of the shaft may be of a dimension that allows the blade and/or tip to proceed from any position that is slightly articulated to the fully articulated position that is at about 90° from the axis that is the shaft length (i.e., about a right angle from the axis that is the shaft length). In other embodiments, full articulation may extend beyond 90° to as far as almost 180° from the first position of being co-linear with the inner lumen of the shaft (i.e., such that the blade is pointing proximally, towards the direction from which the curette has been deployed). Other embodiments may allow articulation to about 60 degrees. Although the slot should be large enough to accommodate the protrusion of the tip and/or blade when the tip and/or blade is articulated, the slot should not be so large as to allow the premature splaying of the tip and/or blade when the tip and/or blade is being moved to the surgical site.

The aperture on the distal end of the shaft may be of a size such that the width of the aperture is slightly wider than the width of the blade. The length of the aperture may be as long as the blade, longer than the blade, or shorter than the blade. Thus, in certain embodiments the aperture may range from 0.5-25 mm in width and/or length, or from about 1-15 mm in width or length.

As described above, the handle at the proximal end of the shaft may contain a means device for remotely articulating the blade and/or tip at or near the distal end of the shaft. In one embodiment, the means device may comprise a track wheel which is connected to an actuating element (for example, a wire or a rod) that runs the length of the shaft in the lumen to a connector piece that directly connects to the proximal side of the blade and/or tip. In an embodiment, the connector piece that connects to the blade comprises a hinge. Alternatively or additionally, the connector piece may comprise no moving parts. The actuating element may be connected directly or indirectly to the connector piece, which is in turn connected to the tip and/or blade. For example, the connector piece may comprise an aperture through which the actuating element (e.g. a wire) may be inserted. These connections may allow the actuating element to control the articulation of the tip and/or blade. For example, in an embodiment, the track wheel may be manipulated by the user by turning the wheel in a given direction to either shorten or lengthen the actuating element (e.g., wire), which in turn can cause the tip and/or blade to articulate.

In an embodiment, the lumen may also optionally contain a guide that keeps the actuating element (e.g., a wire) in place in the lumen of the shaft such that the actuating element maintains a substantially straight orientation with respect to the shaft. Additionally, the guide may prevent the actuating element (for example, a wire) from shearing. Also, the guide may add strength to the blade, as the blade may comprise a torsional stress due to the force applied to the blade when it is in a position that is at least partially outside the shaft and being used to scrape or push against a solid body part. The guide may run for a portion of the lumen, or may comprise almost the entire lumen of the shaft.

In an embodiment, the track wheel of the handle has sufficient resistance so that when the track wheel is manipulated to set the actuating element at a desired position and thereby articulate the tip and/or blade, the actuating element remains in that desired position, even if torsion (such as the force resulting from using the blade to scrape a body part) is applied upon the tip and/or blade.

In alternate embodiments, the tip and/or blade may be made of a material that is satisfactory for its surgical purpose (i.e., scraping and or scoring). These materials may include stainless steel, various nickel-titanium alloys (such as NITINOL) as well as other metal alloys that may be suitable for surgical purposes. In alternative embodiments, the tip and/or blade may be constructed of hard plastics that are reusable or alternatively, may be disposable.

Although the curette of the instant invention has been described as containing only one blade and/or tip, it is within the scope of the invention to have several blades and/or tips that can protrude from the slot when articulated, or alternatively, the plurality of blades and/or tips can protrude through any of a plurality of slots when articulated.

The size of the blades may range as is required by the surgical procedure. Also, whereas in some cases a single blade size may suffice for the procedure, in other embodiments, a plurality of blades may be used. Thus, in certain non-limiting embodiments, the blades used may range from 0.5-25 mm in width and/or length, or from about 1-15 mm in width or length.

In an embodiment of the invention, the shaft contains a cap or a circular ring at the distal end of the shaft. The cap may be positioned at the distal end of the shaft so as to allow easy passage of the curette to the surgical site. The cap may perform several functions. First, the cap may protect the tip and/or blade, so as to prevent the tip and/or blade from splaying when the distal end of the curette is being positioned at the surgical site. Also, the cap (and/or ring) may constrain the distal end of the shaft and provide structural stability. Third, in an embodiment, the cap may serve as a blunt needle (e.g., the cap may be configured with a tapered profile) allowing the curette to be deployed through obstructions as the curette passes to the surgical site. In this embodiment, the cap may be sharpened to facilitate passage of the curette through skin, tissue or other matter to arrive at the surgical site.

The distal cap at the end of the shaft may be made of plastic. In an embodiment, the cap may be disposable. Alternatively to a plastic cap, it is contemplated and therefore within the scope of the invention to have a cap made of other materials, such as polymeric materials, rubbers, metals, and any other material that renders the cap suitable to fit on the end of the shaft. The cap may be fixed securely on the end of the shaft so that when it is being positioned at the surgical site, it does not fall off. In an embodiment, the cap may be fixedly secured on the distal end of the shaft. For example, a glue or any of a variety of epoxides may be used to securely fix the cap on the shaft. Alternatively, the cap may not be secured with glue but may be manufactured so as to fit snugly over the OD of the shaft so that removal is not likely during deployment of the curette. In this embodiment, the cap may be removed to exposed the blade and/or tip actuation means.

In other embodiments, the curette may be designed so as not to have a cap at the end of the shaft, but to comprise a blunt, smoothly finished distal end that is a continuation of the shaft. In this embodiment, the blunt smoothly finished distal end may be made of the same material from which the shaft is made. Materials used for the end of the shaft may include stainless steel, nickel-titanium alloys (such as NITINOL), other metal alloys, and hard polymeric plastics.

The cap or the end of the shaft may also have a small hole in the center or not in the center of the cap to form a ring type structure that allows passage of an accessory device through the shaft to the distal end of the shaft, and through the cap. In alternate embodiments, the accessory device may comprise a needle, another surgical instrument that is smaller in diameter than the hole, or fiber optics from the proximal end of the shaft.

In one embodiment, the curette may comprise a working cannula. For example, in certain cases, the lumen of the curette may have all of the inner components removed while allowing the shaft to remain in position. Thus, in alternate embodiments, the blade (tip), and/or the actuating element, and/or the guide and any other components that are inside the shaft may be removed. When the inner components of the curette are removed, the shaft may then serve the purpose of a cannula. This may be advantageous in that additional surgical procedures can be performed without having to remove the shaft of the curette. By not having to remove the curette shaft, there may be, in certain embodiments, less trauma to the patient. Additionally, by not removing the shaft, the time needed to perform the surgical operation may be reduced. For example, the passage of optical fibers (or other accessory devices such as an endoscope) through the shaft while the shaft is in position at the surgical site may allow a surgeon to view the surgical site to determine if additional scoring and/or scraping to create a cavity is needed. If additional scoring and/or scraping is needed, the optical fibers can be removed and the inner components of the curette returned to continue forming the cavity.

In one embodiment of the instant invention, the curette may comprise a ramp or sloped surface to guide positioning of the curette tip or blade. The ramp may be located inside of the lumen at the distal end of the shaft and proximate to the cap. The ramp may facilitate the articulation of the blade and/or tip from a position that is inside and parallel to the shaft to a position that is not parallel to and substantially outside the shaft. In an embodiment, as the blade is urged distally (e.g. using the actuation device), the blade may engage the ramp and be guided by the surface of the ramp through the slot to proceed outside of the lumen. The ramp can be made of any of a plurality of materials including stainless steel, nickel-titanium alloys (such as NITINOL), other metal alloys, polymeric materials, rubber, or any other material that is structurally suited to facilitating the articulation of the blade and/or tip. The ramp which engages the cutting tip 4 can be designed with any of a plurality of various angles, such that when the cutting tip (e.g., the pick, blade and/or tip) is fully deployed it will be angled at any of a plurality of various angles in relation to the elongated axis of the shaft. For example, the cutting tip, when fully deployed may be at, for example, an angle of 50 degrees, 60 degrees, 70 degrees, 80 degrees, 90 degrees or any other angle relative to the shaft. In variations of the present invention, the surface of the ramp for engaging the cutting tip may have built-in curvature wherein the ramp may be either concave or convex. Embodiments of the present invention may be directed to surgical instruments and methods for creating cavities in body regions.

In one embodiment, the present invention comprises a cavity-creating surgical instrument comprising a cylindrical shaft, the shaft having a lumen inside the shaft, the shaft also having a proximal end and a distal end, the proximal end of the shaft connected to a handle and the distal end connected to a cap, wherein at or near the distal end is a slot that runs essentially parallel to the shaft length wherein a blade, pick, or tip can articulate to or from a position that is in the lumen and substantially parallel to a length of the shaft to any of a plurality of positions through the slot that are at least partially outside and not parallel to the length of the shaft. In an embodiment, the shaft comprises a ramp (e.g., sloped surface) at the distal end of the lumen. In an embodiment, as the blade is urged distally (e.g. using an actuation device), the blade (pick or tip) rotates and may engage the ramp and be guided by the surface of the ramp through the slot to proceed outside the lumen.

In another embodiment, the present invention comprises a method of creating a cavity in a body region comprising presenting a surgical instrument at a surgical site, the surgical instrument comprising a cylindrical shaft, the shaft having a lumen, a proximal end, and a distal end, where the proximal end of the shaft is connected to a handle and the distal end is connected to a cap, and where the shaft has a slot at or near the distal end that runs essentially parallel to the shaft length, wherein a blade, pick, or tip occupies a position that is at least partially outside of the lumen and not parallel to the length of the shaft; and rotating the shaft by the handle can score or scrape a body region to create a cavity in said body region. In an embodiment, the shaft comprises a ramp (e.g., a sloped surface) at the distal end of the lumen. In an embodiment, as the blade is urged distally (e.g. using an actuation device), the blade (pick or tip) may engage the ramp and be guided by the surface of the ramp through the slot to outside of the lumen.

In yet another embodiment, the present invention comprises a kit comprising a cavity-creating surgical instrument of the present invention. In an embodiment, the cavity-creating surgical instrument may comprise a cylindrical shaft, the shaft having a lumen inside the shaft, the shaft also having a proximal end and a distal end, the proximal end of the shaft connected to a handle and the distal end connected to a cap, wherein at or near the distal end is a slot that runs essentially parallel to the shaft length wherein a blade, pick, or tip can articulate to or from a position that is in the lumen and substantially parallel to a length of the shaft to or from any of a plurality of positions through the slot that are at least partially outside and not parallel to the length of the shaft. In an embodiment, the shaft comprises a ramp (e.g., sloped surface) at the distal end of the lumen. In an embodiment, as the blade is urged distally (e.g. using an actuation device), the blade (pick or tip) may rotate and/or engage the ramp and be guided by the surface of the ramp through the slot to outside the lumen. The kit may further comprise an access cannula.

The surgical instrument of the present invention may have a cutting element (e.g., blade, pick, or tip) that is remotely articulated by a device on the handle that can be manually manipulated. The blade, pick, or tip in the surgical instrument may be optionally articulatable from an angle that may range from 0 degrees to almost 180 degrees, or from about 5 degrees to about 90 degrees, or from about 5 degrees to about 60 degrees. In an embodiment, the shaft comprises a ramp (e.g., sloped surface) at the distal end of the lumen. In an embodiment, as the blade is urged distally (e.g. using an actuation device), the blade (pick or tip) may engage the ramp and be guided by the surface of the ramp through the slot to outside of the lumen. Moreover, the surgical instrument of the present invention may have a means such as a wire that can be used to articulate the blade, pick, or tip. To guide the wire, the surgical instrument may also comprise a wire guide. The wire guide may occupy any portion in the volume of the shaft. In one embodiment, the wire guide is present in the lumen in the distal portion of the shaft and the wire guide may occupy a portion that is less than or equal to about half the lumen volume of the shaft.

Using a curette for scoring or scraping may put a torsional force on the blade and parts of the curette connected to the blade (for example, the actuating element). Thus, in an embodiment, a guide for the actuating element may be made of a polymeric material that has deformation qualities. Using a material that is resilient to deformation may reduce shearing of the wire when a torsional force is applied upon the blade, tip, or pick. Further, should the wire guide become deformed to some degree by the torsional force applied upon it by the wire, the wire guide can be removed from the shaft lumen by pulling on the wire and have the blade, tip, or pick act as a barb.

The blade, tip, or pick may, in certain embodiments, be actuated by a rod that is adjacent to the handle at the proximal end of the shaft. The rod actuation may comprise a ball and socket at the distal end that allows the articulation of the blade, tip, and/or pick to the desired position. In this embodiment, the rod may be pushed or pulled by an actuation means on the handle to thereby modify the amount of articulation on the blade, tip, or pick. In other embodiments, the present invention also comprises using hydraulics to actuate the blade, tip and/or pick. In yet another embodiment, an electromechanical or magnetic linkage between the blade and the actuation device may be used to articulate the blade, pick and/or tip. In an embodiment, a wire not only provides a good actuation device but the wire also adds strength to the blade, tip or pick.

The surgical instrument of the present invention, and its component parts may be made of any of a plurality of materials. In an embodiment, the shaft may be made of a nickel titanium alloy. Also in an embodiment, the shaft may have a diameter that is substantially uniform through the length of the shaft. In an embodiment, the shaft may comprise an outer diameter that is in the range of about 0.25 to 0.35 cm or alternatively, it may be larger or substantially larger.

In an embodiment, the surgical instrument may have a cap or other enclosure at the distal end. Also in an embodiment, the cap may be removable. Alternatively, the cap may be glued to the distal end of the shaft. The cap may or may not have a hole in it.

As described above, in another embodiment, the present invention relates to a method of creating a cavity in a body region comprising presenting a surgical instrument at a surgical site, the surgical instrument comprising a cylindrical shaft, the shaft having a lumen, a proximal end and a distal end, where the proximal end of the shaft is connected to a handle and the distal end is connected to a cap, and further having a slot at or near the distal end that runs essentially parallel to the shaft length, wherein a blade, pick, or tip occupies a position that is at least partially outside the lumen and not parallel to the length of the shaft; and rotating the shaft by the handle to score or scrape the body region, to create a cavity in said body region. In an embodiment, the shaft comprises a ramp (e.g., sloped surface) at the distal end of the lumen. In an embodiment, as the blade is urged distally (e.g. using an actuation device), the blade (pick or tip) may engage the ramp and be guided by the surface of the ramp through the slot to outside of the lumen. The kit may further comprise an access cannula.

In the above enumerated method, the blade, pick, or tip in the surgical instrument may be optionally articulatable from an angle that may range from 0 degrees to almost 180 degrees, or from about 5 degrees to about 90 degrees, or from about 5 degrees to about 60 degrees.

In certain embodiments, the blade, pick, or tip can be made of any of a plurality of materials such as diamond, quartz, graphite, carbon nanotubes, stainless steel, or any of a plurality of metal alloys such as a nickel titanium alloy, or a cobalt alloy, or any combination of these materials. In an embodiment, nickel titanium alloy and stainless steel are used. In choosing the materials used, it is recognized that the blade, tip, or pick may have sufficient strength to perform the desired function (i.e., scoring or scraping) and also be biocompatible (so that infection or other complications do not occur to the patient). In an embodiment, the method further comprises optionally passing the surgical instrument through a cannula.

The methods and devices of the present invention may be used for a variety of surgical procedures. In an embodiment, the cavity-creating device is used on bone. For example, in an embodiment, the bone is a vertebral bone.

In an embodiment, the surgical instrument while being presented at the surgical site is not passed through a cannula. For example, the inner components of the curette can be removed and the shaft of the curette can serve as the cannula. The method may further include the step of checking the surgical site with an endoscope to see if sufficient scoring and/or scraping has occurred.

In yet another embodiment, the present invention comprises a method of making a cavity-creating device. In an embodiment, the method may comprise making a tubular shaft. For example, the shaft may be made by drawn extrusion for a plastic part, or rolled and welded for a metal part. The method may further comprise attaching a containing ring to the distal guide and attaching the distal guide to the lumen. In another embodiment, the distal guide may comprise the entire lumen. The method may further comprise directing a wire through a hole on the proximal end of the blade and feeding the wire through the lumen of the shaft. The blade and wires may then be aligned with the axis of the lumen using the distal guide. Next the wire may be connected to the actuation mechanism and the tension adjusted such that the blade is parallel to the lumen when the wire is tightened towards the proximal end of the shaft and perpendicular to the lumen when the wire is extended towards the distal end of the shaft. In an embodiment, 20 pound wire may be used. In an embodiment, the method further comprises furnishing a sloped surface as the distal surface in the lumen of the shaft. Also, a cap or other type of enclosure may be fixed to the distal end, for example, either by welding or an adhesive.

Having described the curette of the instant invention in general, the curette of the present invention is described with reference to the figures. It should be understood that these figures are not to limit the present invention but are rather illustrative of one embodiment of the present invention.

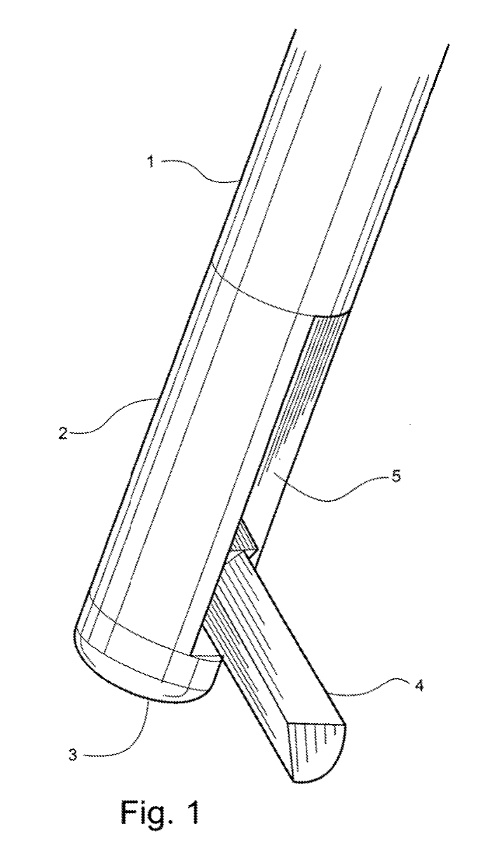

FIG. 1 shows a perspective 3D view of a curette that exemplifies one embodiment of the present invention. In this view, a shaft 1 contains a distal end of the shaft 2 that contains a slot 5 that allows the tip and/or cutting element 4 (e.g., a blade, tip, or pick) to articulate so that the tip and/or blade goes from a position that is inside the shaft 1 (and 2) to a position that is at least partially outside the shaft (1 and 2). In alternative embodiments the outer diameter (OD) of the shaft may comprise a diameter of about 0.1 to 2.5 cm, or from about 0.1 to 0.5 cm, or from about 0.2 to 0.4 cm in diameter. In one embodiment, the diameter may range from about 0.109 inches (size 2=˜0.28 cm) to 0.134 inches (size 3=˜0.35 cm). Also, the shaft length may range from about 20 to 40 cm, or from about 25 to 35 cm, or from 25 to 30 cm.

The blade and/or tip 4 is attached to an actuating element (not shown in this figure) that goes through the lumen of the shaft (1 and 2) and allows the blade and/or tip 4 to articulate from a position that is entirely inside the shaft (1 and 2) to a position that is at least partially outside the shaft. In alternate embodiments, the tip and/or blade may be made of a material that is satisfactory for its surgical purpose (i.e., scraping and or scoring). These materials may include stainless steel, various nickel-titanium alloys (such as NITINOL) as well as other metal alloys that may be suitable for surgical purposes. In alternative embodiments, the tip and/or blade may be constructed of hard plastics that are reusable or alternatively, may be disposable. The blade may vary in size as required by the surgical procedure. Thus, the blades used may range from 0.5-25 mm in width and/or length, or from about 1-15 mm in width or length.

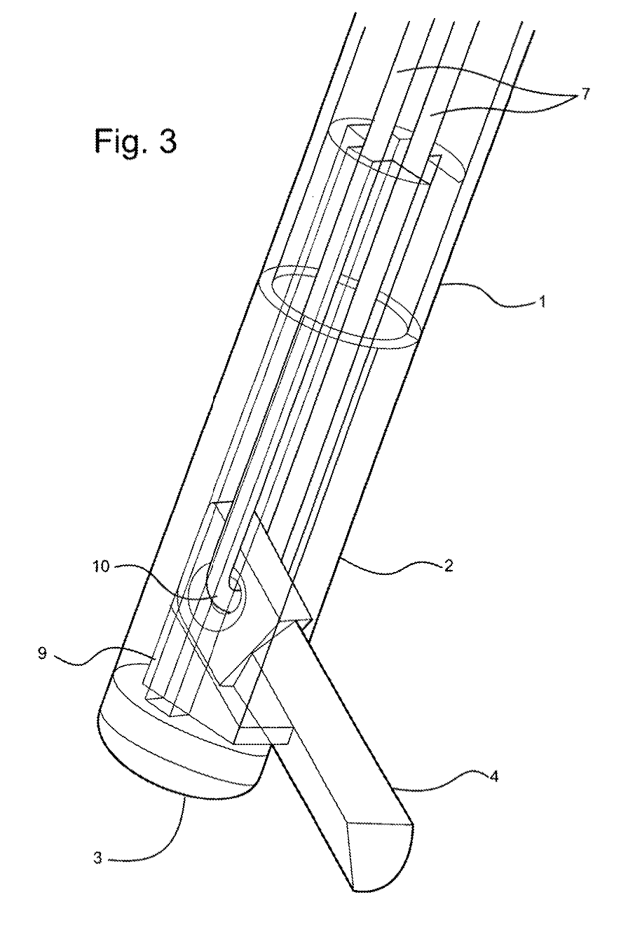

At the very end of the distal end of the shaft 2 is a cap 3. The cap 3 may contain a ramp (not shown in this figure but shown in FIG. 3) that allows the blade and/or tip to deploy through the slot 5. The length of the slot may be as long as the blade, longer than the blade, or shorter than the blade. Thus, in certain embodiments the slot may range from 0.5-25 mm in width and/or length, or from about 1-15 mm in width or length.

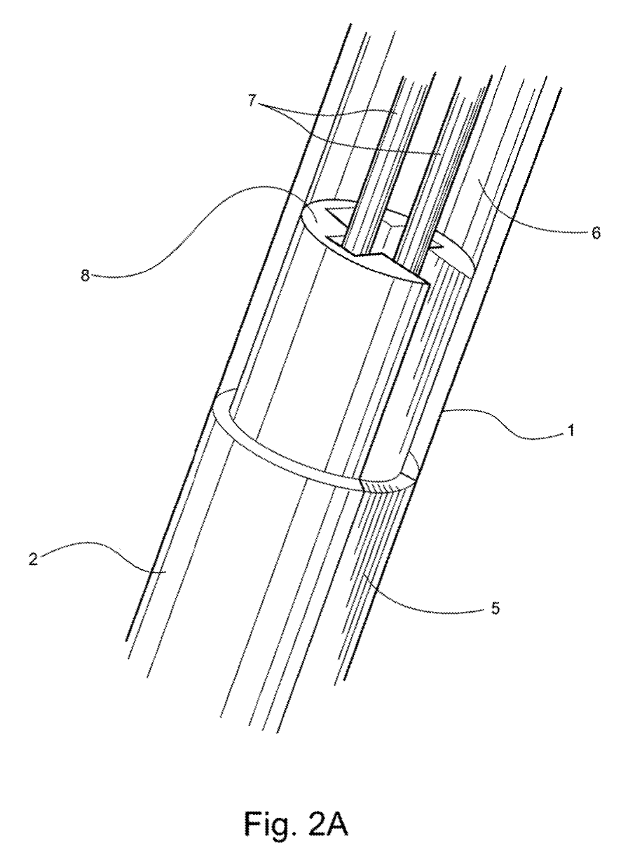

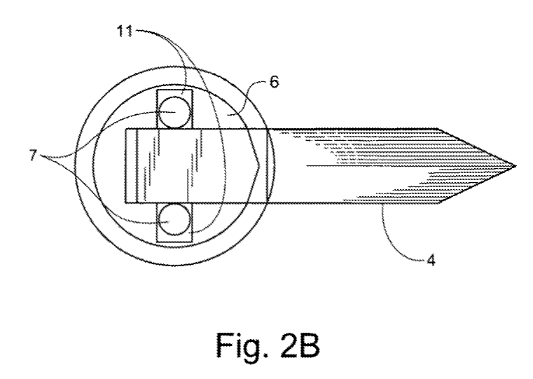

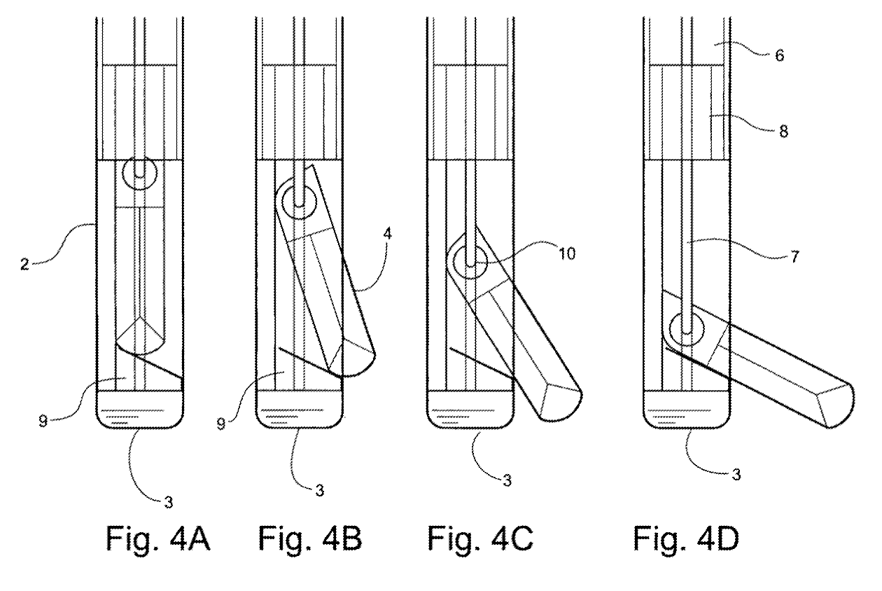

FIGS. 2A and B show a transparent 3D rendering of the lumen of the curette and a cross-sectional top down view of the lumen of the curette, respectively. In FIG. 2A, a slot (side window) 5 is shown that leaves access for the blade and/or tip (not shown in FIG. 2A) to move from a position that is inside and parallel to the length of the shaft 1 (and 2) to a position that is at least partially outside the distal end of the shaft 2. The lumen 6 contains a guide 11 (please note that both 8 and 11 are guides that show slightly different embodiments of the invention) that guides the actuating element 7 that articulates the tip and/or blade (not shown in FIG. 2A) to a position that is entirely inside the shaft (1 and 2) to a position that is at least partially outside the shaft. Those of ordinary skill in the art having the benefit of this disclosure will recognize that the actuating element can be a wire or rod or any of a plurality of other elements that articulate the cutting element 4. The guide 8 also has a slot that aligns with the slot 5 that allows the blade to pass from inside the shaft to outside the shaft. When the actuating element 7 is shortened by the control mechanism on the handle (not shown), the tip and/or blade articulates to a position that is more inside the lumen of the shaft. When the control mechanism on the handle lengthens the actuating element 7 (for example, a wire), the tip and/or blade articulates to a position that is more outside the shaft (see FIGS. 4A-D). In one variation, the profile/channel of the guide (8 in FIG. 2A) at a more proximal location in the curette matches the profile at a more distal end of the cutting element 4 to prevent clockwise or counter clockwise axial movement along the length of the shaft when the cutting element is deployed (i.e., protruding from the shaft). This allows the torque generated by the user at the proximal end of the curette to be transmitted to the tip of the cutting element when the curette is rotated/turned by the user.

FIG. 2B shows a cross sectional top down view of an embodiment of the instant invention. In this figure, the tip and/or blade 4 is seen in its most articulated position so that it is maximally outside the shaft. The actuating element 7 (for example, a wire) is shown on each side of the blade and the actuating element 7 is positioned so that it is guided by the guide 11. In a variation, the wire passes through the tip and/or blade 4 at a hinge 10 (not shown in FIG. 2B but shown in FIG. 3) which articulates the blade to any of a plurality of positions from a position that is parallel to and completely inside the shaft to a position that is substantially outside and axial (for example, perpendicular or 60 degrees) to the shaft.

FIG. 3 shows a transparent 3D rendering of the shaft wherein the blade and the mode of action for moving the blade of the curette is visible. The actuating element 7 moves the blade and/or tip 4 so that the blade and/or tip 4 articulates at a hinge 10 from a position that is inside and parallel to the shaft to a position that is not parallel and at least partially outside the shaft. At the very distal end of the lumen there is a ramp 9 that aids in articulating the blade form the position that is inside and parallel to the shaft to a position that is not parallel and at least partially outside the shaft (see also FIG. 4).

The slanted surface of the ramp which engages the cutting tip 4 may be designed with various angles, such that when the cutting tip (e.g., blade and/or tip) is fully deployed it will be angled at any of a plurality of various angles in relation to the elongated axis of the cannula. In variations of the present invention, the surface of the ramp for engaging the cutting tip has a built-in curvature (either concave or convex).

FIGS. 4A-D show four different transparent 3D views of the blade in the lumen of the curette at different levels of deployment (articulation) of the blade. The actuating element 7 at its most extended position (see FIG. 4D) articulates the blade and/or tip to a position that is substantially outside of the shaft allowing a physician to create a large cavity when the curette is rotated. As the actuating element 7 is shortened the blade and/or tip moves to a position that is more substantially inside the lumen (see for example FIG. 4C when compared to FIG. 4D). When the physician rotates the curette with the blade and/or tip as it appears in FIG. 4C a smaller cavity (from scraping and or scoring) is produced (relative to the blade and/or tip position at FIG. 4D). The ramp 9 which is positioned at the end of the shaft 2 in the lumen 6 facilitates the articulation of the blade and/or tip from a position wherein the blade and/or tip is inside the lumen 6 and parallel to the shaft (FIG. 4A) to a position that is substantially outside and almost perpendicular to the shaft (FIG. 4D). The ramp also facilitates articulation back from the position wherein the blade and/or tip is substantially outside and almost perpendicular to the shaft (FIG. 4D) to a position wherein the blade and/or tip is inside the lumen 6 and parallel to the shaft (FIG. 4A).

FIGS. 4A-D illustrate one embodiment of the invention. In the illustrated embodiments, the guide 8 is shown so that it only occupies part of the lumen 6 of the shaft 1 (see FIG. 4D). It should however be understood that the guide can occupy substantially more of the lumen. It is contemplated and therefore within the scope of the invention that the guide 8 can occupy a substantial part of the lumen from a position that is very close to the proximal end of the shaft (e.g., close to where the handle connects to the shaft) to a position that is close to or in the distal end of the shaft 2.

Thus, the present invention is directed to a cavity creating surgical instrument comprising a cylindrical shaft, the shaft having a lumen inside the shaft, the shaft also having a proximal end and a distal end, the proximal end of the shaft connected to a handle and the distal end connected to a cap, wherein at or near the distal end is a slot that runs essentially parallel to the shaft length wherein a blade, pick, or tip can articulate to or from a position that is in the lumen and substantially parallel to a length of the shaft to any of a plurality of positions through the slot that are at least partially outside and not parallel to the length of the shaft.

In an embodiment, the blade, pick, or tip is remotely articulated by a device on the handle that can be manually manipulated. The device of the instrument may have a wire that is used to articulate the blade, pick, or tip.

The surgical instrument of the present invention may further comprise a ramp that facilitates articulation of the blade, pick, or tip. The surgical instrument of the present invention may further comprise a wire guide. The wire guide may occupy a portion that is less than half the lumen volume of the shaft.

In one embodiment, the surgical instrument of the present invention may have a shaft that is made of a nickel titanium alloy. The shaft may have a substantially uniform diameter through the length of the shaft wherein the shaft has an outer diameter that is about 0.25 to about 0.35 cm in diameter.

The cap at the distal end may be removable, or alternatively, the cap may be glued to the distal end of the shaft. There may be a hole in the cap.

Moreover, in an embodiment, the present invention relates to a method of creating a cavity in a body region comprising: presenting a surgical instrument at a surgical site, the surgical instrument comprising a cylindrical shaft, the shaft having a lumen inside the shaft, the shaft having a proximal end and a distal end, the proximal end of the shaft connected to a handle and the distal end connected to a cap, and a slot at or near the distal end that runs essentially parallel to the shaft length, wherein a blade, pick, or tip occupies a position that is at least partially outside the lumen and not parallel to the length of the shaft; rotating the shaft by the handle to score or scrape the body region, thus creating a cavity in said body region.

In an embodiment, the blade, pick, or tip may be articulatable. In an embodiment, the blade, pick, or tip may be made of a nickel titanium alloy.

In an embodiment, the method of the present invention may further comprise passing the surgical instrument through a cannula. Alternatively, the method of the present invention uses the surgical instrument at the surgical site and is not passed through a cannula. In another alternative, the method comprises using the surgical instrument as the cannula.

In another embodiment, the method of the present invention may have the cavity created in bone and the bone may be a vertebra. For example, the distal portion of the surgical device may be configured for insertion through the pedicle of the vertebra. Once inside the vertebral body, the cutting element may be deployed from the shaft by displacing the actuating element (e.g., rods) which forces the tip portion of the cutting element (e.g., blade) to engage the ramp and protrude through the side window on the shaft.

The method of the present invention may have the surgical site checked with an endoscope to see if sufficient scoring and/or scraping has occurred.

The present invention has been described with reference to examples. However, it should be apparent to those of skill in the art that modifications of the present invention can be made without departing from the spirit and scope of the invention. Further, it is contemplated and therefore within the scope of the present invention that any element that is described anywhere in the above description can be combined with any other element. Moreover, when a range is disclosed, any number that falls within the range is a contemplated endpoint, even if that number is not explicitly disclosed. Where methods and steps described above indicate certain events occurring in a certain order, those of ordinary skill in the art having the benefit of this disclosure should recognize that the ordering of certain steps may be modified and that such modifications are in accordance with the variations of the invention. Additionally, certain of the steps may be performed concurrently in a parallel process when possible, as well as performed sequentially as described above. Furthermore, a claim limitation should be interpreted to invoke 35 U.S.C. 112, sixth paragraph, if and only if the claim limitation use the phrase(s) “means for” or “step for.” A claim element that does not include the phrase “means for” or “step for” should not be considered to invoke 35 U.S.C. 112, sixth paragraph. In any event, the breadth and scope of the invention should not be limited by any of the above-described embodiments, but should be defined in accordance with the following claims.

We claim:

1. A method of creating a cavity in a body region comprising:
inserting a distal portion of a shaft into a patient's body;
rotating a cutting element within a lumen of the shaft which results in a distal portion of the cutting element protruding from a side window on the shaft, wherein the side window is located on a circumferential surface at a distal portion of the shaft; and
rotating the shaft axially to score or scrape a body region within the patient's body to create a cavity.

2. The method according to claim 1, further comprising:
displacing the cutting element distally within the lumen of the cannula.

3. The method according to claim 2, further comprising having the cutting element engage a ramp.

4. The method according to claim 1, wherein inserting the distal portion of the shaft comprises inserting the distal portion of the shaft into a vertebral body through a pedicle.

5. The method according to claim 1, wherein the cutting element is a blade, tip or pick and the blade, pick, or tip is articulatable.

6. The method according to claim 5, wherein the blade, pick, or tip is made of a nickel titanium alloy.

7. The method according to claim 5, further comprising passing the shaft through a cannula.

8. The method according to claim 5, wherein the cavity is created in bone.

9. The method according claim 8, wherein the bone is a vertebra.

10. The method according to claim 5, wherein the shaft is not passed through a cannula.

11. The method according to claim 10, wherein the shaft also serves as a cannula.

12. The method according to claim 11 further comprising checking a surgical site with an endoscope to see if sufficient scoring and/or scraping has occurred.
